(19) United States
(12) Patent Application Publication
(10) Pub. No.: US 2011/0034690 A1
(43) Pub. Date: Feb. 10, 2011

(54) PROCESS FOR THE PREPARATION OF PURE PRULIFLOXACIN

(75) Inventors: Tarun Kant Sharma, Gurgaon (IN); Raghuram Morampudi, Krishina (IN); Shanmugam Srinivasan, Hyderabad (IN)

Correspondence Address: Ranbaxy Inc., Intellectual Property Department, 600 College Road East, PRINCETON, NJ, 08540, US

(73) Assignee: RANBAXY LABORATORIES LIMITED, Gurgan, Haryana (IN)

(21) Appl. No.: 12/531,255

(22) PCT Filed: Mar. 14, 2008
(86) PCT No.: PCT/IB2008/050972

§ 371 Date: Oct. 27, 2010

(30) Foreign Application Priority Data
Mar. 14, 2007 (IN) 545/DEL/2007

(51) Int. Cl. C07D 401/14

(52) U.S. Cl. 544/361

(57) ABSTRACT

The present invention relates to a process for the preparation of prulifloxacin. The present invention further relates to prulifloxacin having purity of about 99% or above.

### FIELD OF THE INVENTION

The present invention relates to a process for the preparation of prulifloxacin. The present invention further relates to prulifloxacin having purity of about 99% or above.

### BACKGROUND OF THE INVENTION

Prulifloxacin is chemically 6-fluoro-1-methyl-7-4-[(5-methyl-2-oxo-1,3-dioxol-4-yl) methyl]piperazin-1-yl{-4-oxo-4H-[1,3]-thiazeto[3,2-a]-quinoline-3-carboxylic acid of Formula I having the structure as depicted below:

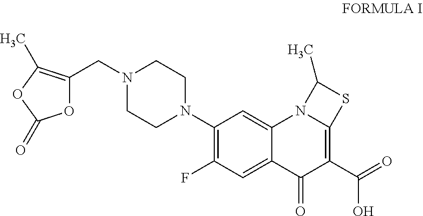

Prulifloxacin has significant antibacterial activity and has been marketed as a synthetic antibacterial agent. U.S. Pat. No. 5,086,049 provides a process for the preparation of prulifloxacin by reacting 6-fluoro-1-methyl-4-oxo-7-piperazin-1-yl-4H-[1,3]thiazeto[3,2-a]quinoline-3-carboxylic acid of Formula II,

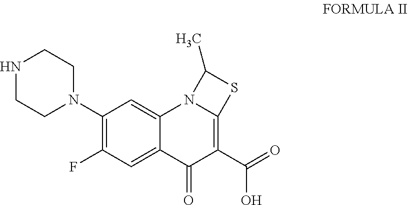

and 4-(bromomethyl)-5-methyl-1,3-dioxol-1-2-one of Formula III,

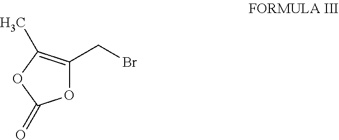

using N,N-dimethylformamide as a solvent. 4-(Bromomethyl)-5-methyl-1,3-dioxol-2-one of Formula III is used in excess to one mole of the compound of Formula II. The process provided in U.S. Pat. No. 5,086,049 further involves concentrating the reaction mixture, pouring the residue into water and isolating prulifloxacin by filtration. The resulting prulifloxacin is recrystallized from chloroform-methanol.

However, U.S. Pat. No. 5,086,049 does not provide any method to remove the unreacted or the excess of 4-(bromomethyl)-5-methyl-1,3-dioxol-2-one of Formula III used as a starting material. The present inventors have observed that it is difficult to obtain prulifloxacin with pharmaceutically acceptable purity by following the process provided in U.S. Pat. No. 5,086,049, which is typically contaminated by process related impurities including 4-(bromomethyl)-5-methyl-1,3-dioxol-2-one.

### SUMMARY OF THE INVENTION

The present inventors have developed a process for the preparation of prulifloxacin which significantly reduces process-related impurities. The present process includes the extraction of prulifloxacin in the aqueous layer in the form of its acid addition salt, and thereby facilitates the removal of organic soluble impurities including unreacted or excess 4-(bromomethyl)-5-methyl-1,3-dioxol-2-one of Formula III. Thus, by employing processes of the present invention, the impurities including 4-(bromomethyl)-5-methyl-1,3-dioxol-2-one can be reduced to an amount of less than about 1% and prulifloxacin can be obtained with a purity of about 99% or above.

### DETAILED DESCRIPTION OF THE INVENTION

In a first aspect, a process for the preparation of prulifloxacin is provided, the process comprising:

a) reacting a compound of Formula II with a compound of Formula III to obtain prulifloxacin;

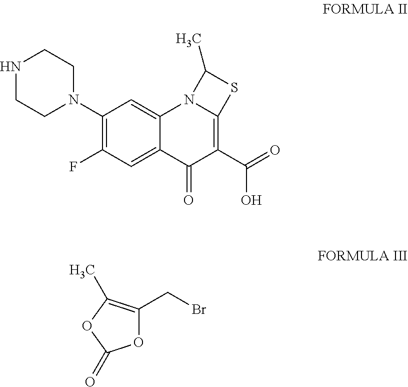

b) contacting the prulifloxacin obtained in step a) with an acid in a biphasic solvent system, wherein the biphasic solvent system comprises water and a water-immiscible organic solvent;

c) separating the aqueous layer from the reaction mixture obtained in step b);

d) treating the aqueous layer with a base; and

e) isolating prulifloxacin.

The process described in steps b-e above may be carried out with prulifloxacin made from any process however.

The compounds of Formula II and Formula III may be prepared according to the methods provided in U.S. Pat. No. 5,086,049. The compounds of Formula II and Formula III are reacted in the presence of an organic solvent and a base. The amount of compound of Formula III is equimolar or excess to one mole of the compound of Formula II. The compound of Formula III may be used in excess to one mole of the compound of Formula II. The organic solvent may be selected from, for example, N,N-dimethyl formamide, dimethylsulfoxide and diglyme. The base may be, for example, an alkali metal carbonate. The reaction can be effected by stirring the reaction mixture at from about 0° to about 50° C. After the completion of the reaction, prulifloxacin is separated from the reaction mixture. The separation may be carried out by pouring the reaction mixture into water and filtering prulifloxacin as a solid. The prulifloxacin so obtained is dissolved in a water-immiscible organic solvent. The water-immiscible organic solvent can be selected from, for example, chloroform, cyclohexane, dichloromethane, 1,2-dichloroethane, diethyl ether, hexane, pentane, methyl-t-butyl ether, ethyl acetate, carbon tetrachloride or the like. The water-immiscible organic solvent can also be a mixture of the foregoing with a C₁₋₃ alkanol. The water-immiscible organic solvent containing prulifloxacin is treated with water. The biphasic reaction mixture so obtained is treated with an inorganic or an organic acid. Alternatively, the water immiscible organic solvent containing prulifloxacin is treated with an aqueous solution of an inorganic or an organic acid. Hydrochloric acid and hydrobromic acid can be used as the acid, for example. The aqueous layer is subsequently separated from the reaction mixture and treated with an inorganic or an organic base to precipitate prulifloxacin as a free base. An inorganic base selected from a group consisting of alkali metal carbonates or hydroxides, and alkaline earth metal carbonates or hydroxides can be used.

The precipitated solid prulifloxacin can be isolated from the aqueous layer by further layer separation. The aqueous layer containing precipitated prulifloxacin is treated with a water-immiscible organic solvent. The water-immiscible organic solvent can be selected from, for example, chloroform, cyclohexane, dichloromethane, 1,2-dichloroethane, diethyl ether, hexane, pentane, methyl-t-butyl ether, ethyl acetate, carbon tetrachloride or the like. The water-immiscible organic solvent can also be a mixture with a C₁₋₃ alkanol. The organic layer is separated from the reaction mixture and the solvent is removed by concentration to obtain prulifloxacin as a solid. Alternatively, the precipitated solid prulifloxacin can be directly isolated from the aqueous layer by filtration.

The prulifloxacin so obtained is further recrystallized to obtain prulifloxacin having purity of about 99% or above. The recrystallization can be carried out from a mixture of a water-immiscible organic solvent and a C₁₋₃ alkanol, for example, a mixture of chloroform and ethanol.

In a second aspect, prulifloxacin having purity of about 99% or above is provided.

In a third aspect, a pharmaceutical composition comprising prulifloxacin having purity of about 99% or above, and optionally containing one or more excipients and/or diluents is provided.

In a fourth aspect, a method of treating bacterial infections in humans and animals which comprises administering to human or animal in need thereof an antibacterially effective amount of prulifloxacin having purity of about 99% or above is provided.

While the present invention has been described in terms of its specific embodiments, certain modifications and equivalents will be apparent to those skilled in the art and are intended to be included within the scope of the present invention.

### Example 1

### Process for the Preparation of Prulifloxacin

Step A): A solution of 4-(bromomethyl)-5-methyl-1,3-dioxol-2-one (35.5 g, 0.184 mole) in N,N-dimethylformamide (200 ml) was added dropwise at 0° to 5° C. to a stirred solution of 6-fluoro-l-methyl-4-oxo-7-piperazin-l-yl-4H-[1,3]thiazeto[3,2-a]quinoline-3-carboxylic acid (50 g, 0.143 mole and potassium bicarbonate (15.8 g, 0.1578 mole) in N,N-dimethylformamide (200 ml). The resulting mixture was stirred at 25° to 28° C. for 3 to 4 hours. After the completion of the reaction, the reaction mixture was poured into water (1250 ml). The solid obtained was filtered, washed with water (100 ml), and subsequently dissolved in a mixture of chloroform: methanol (7:3; 1250 ml). The lower organic layer was separated and water (500 ml) was added to the organic layer. A dilute aqueous solution of hydrochloric acid was added to the biphasic reaction mixture to adjust pH to 0.8 to 1.0. The reaction mixture was stirred for 15 minutes, allowed to settle and the upper aqueous layer was separated. The process was repeated twice and the aqueous layers were combined. Activated charcoal (10%) was added to the combined aqueous layer and stirred for 30 minutes, filtered and cooled to 20° to 25° C. The pH of the reaction mixture was adjusted to 6.5 to 7.0 by adding an aqueous solution of sodium bicarbonate. The solid obtained was extracted with chloroform (375 ml), stirred for 15 minutes and the organic layer was separated. The aqueous layer was further extracted with a mixture of chloroform: methanol (7:3 ratio; 50 ml). The combined organic layer was distilled under vacuum at 35° to 40° C. to recover the solvent up to 125 ml. The reaction mass so obtained was stirred for 3 to 4 hours at 28° to 30° C., filtered and washed with chilled chloroform (50 ml). The wet cake obtained was dried at 45° C. for 12 hours to obtain the title compound.
Step B): The prulifloxacin (30 g) obtained in Step A) was suspended in a mixture of chloroform: ethanol (10:1, v/v, 585 ml: 58.5 ml) and heated to reflux temperature. Activated carbon (3.9 gm) was added to the partially cleared solution and refluxed for 30 minutes, followed by filtration through Celite bed. The bed was further washed with chloroform: ethanol (10:1, v/v, 585 ml: 58.5 ml). The filtrate so obtained was distilled at atmospheric pressure till to partially remove the solvent. The concentrate so obtained was stirred at about 25° C. for 1 hour, and filtered. The solid obtained was washed with chloroform: ethanol (39 ml×2), dried under vacuum at 45° C. for 12 hours to obtain the title compound.

### Yield: 22 g

HPLC Purity: 99%

We claim:

1. A process for the preparation of prulifloxacin, wherein said process comprises,
a) contacting prulifloxacin with an acid in a biphasic solvent system, wherein the biphasic solvent system comprises water and a water-immiscible organic solvent,
b) separating the aqueous layer from the reaction mixture obtained in step b)
c) treating the aqueous layer with a base, and
d) isolating prulifloxacin from the reaction mixture thereof.

2. A process according to claim 1, wherein the water-immiscible organic solvent of step b) is selected from a group consisting of chloroform, cyclohexane, dichloromethane, 1,2-dichloroethane, diethyl ether, hexane, pentane, methyl-t-butyl ether, ethyl acetate and carbon tetrachloride.

3. A process according to claim 1, wherein the acid is hydrochloric acid or hydrobromic acid.

4. A process according to claim 1, wherein the base is selected from a group consisting of alkali metal carbonates or hydroxides, and alkaline earth metal carbonates or hydroxides.

5. A process according to claim 1, wherein the prulifloxacin obtained in step d) is further recrystallized from a mixture of a water immiscible organic solvent and a C₁₋₃ alkanol.

6. A process according to claim 5, wherein the water-immiscible organic solvent is chloroform.

7. A process according to claim 5, wherein the C₁₋₃ alkanol is ethanol.

8. Prulifloxacin having purity of about 99% or above.
